(19) United States
(12) Patent Application Publication
(10) Pub. No.: US 2022/0287915 A1
(43) Pub. Date: Sep. 15, 2022

(54) System and Method for Robotic Pharmacy-Filled, Wirelessly Enabled Pillbox with Configurable Weekly and Monthly Modes

(71) Applicant: PILLSY, INC., Seattle, WA (US)

(72) Inventor: Jeffrey LeBrun, Seattle, WA (US)

(73) Assignee: Optimize Health, Inc., Seattle, WA (US)

(21) Appl. No.: 17/630,162

(22) PCT Filed: Jul. 25, 2020
(86) PCT No.: PCT/US20/43642

§ 371 Date: Jan. 25, 2022

Related U.S. Application Data
(60) Provisional application No. 62/878,952, filed on Jul. 26, 2019.

(51) Int. Cl. A61J 7/04; A61J 1/03; A61J 7/00; G16H 20/10

(52) U.S. Cl. CPC A61J 7/0436; A61J 1/03; A61J 7/0069; A61J 7/0418; G16H 20/10; A61J 2200/30; A61J 2200/70; A61J 2205/60; A61J 2205/00; A61J 2205/30

(57) ABSTRACT

A smart pill organizer device for use in a networked computer system to wirelessly monitor a patient's adherence to medication doses, sends alerts when missed, and order refills as needed. The device is a container comprising: a tray of removable dose units tagged with a unique identifier assigned to a patient. The tag may comprise a scannable barcode, and the scanner may be part of the device, or kit. The device further comprises computer components and wireless transmitters, and one or more electromagnetic sensors, e.g. positioned beneath the dose units, to detect when a dose is taken, and transmit the information to a remote server comprising a patient database record. When a dose is missed, an alert is sent to one or more third party electronic computing devices (e.g. patient's, doctor's, pharmacy, caregiver's, etc.). The dose units are filled automatedly with a patient's prescription by a pharmaceutical robotic system.

### CROSS-REFERENCE TO RELATED APPLICATIONS

This is a § 371 National Stage application of International Patent Application PCT/US2020/043642 filed on Jul. 25, 2020, which claims the benefit of U.S. provisional Ser. No. 62/878,952 filed on Jul. 26, 2019, the entire contents of which are hereby incorporated by reference in its entirety.

### FIELD OF THE INVENTION

The present invention relates to containers for holding medications, with marked sections for each day or dose; and in particular to smart pill containers that wirelessly communicate with remote servers/computers to track a patient's adherence to a dosing schedule.

### BACKGROUND OF THE INVENTION

Medication non-adherence is a problem that has been said to cost the U.S. healthcare system over 125,000 lives, and $300 billion each year. Containers comprising pill organizers have been shown to be an effective way to help people who have multiple medications organize their medications; however, errors often occur when people fill their own pillboxes. Most pharmacies will not fill pill organizers due to the lack of a scalable way of doing so.

Remote monitoring of medications dosing has also been shown to be an effective way to improve medication adherence. While current designs for remote sensing of medication boxes exist, there is a need within the pharmacy industry for an automated-robotic system to fill smart-wireless pillboxes.

Other issues with existing designs for wireless pillboxes include individual units that are too small to hold the right quantity of pills; or that are too large to be fillable by a robotic system; or that are connected in a way that makes them difficult or impossible to be filled in an automated manner.

Additionally, mechanical or light-based sensing methods do not reliably detect the removal of a dose under a range of conditions. Other sensor configurations consume a large amount of battery life, making them impractical.

Described herein is a smart, wireless pillbox, that can be easily automatedly, robotically filled and labeled at the pharmacy, customizable to each patient's medication intake, and which requires a significantly lower amount of battery power than the prior art to detect when the doses are administered.

### SUMMARY OF THE INVENTION

The present invention comprises a smart pill organizer storing a plurality of a patient's medication dose units; method of using in a networked system for monitoring dose adherence by a remote computer; a computer program product; and a method of forming and filling the dose units using a plastic sheet in a robotic machine that is filled with the medications and then cut into the individual dose units.

The smart pill organizer device may also be pre-programmed with scheduled doses. If a dose is taken within a pre-programmed dose interval, then the dose will be automatically recorded and a reminder will not occur. If the dose is not taken, then the organizer device will emit a reminder; or if the wrong dose is taken, an alert is sent. Optionally, a mobile application of the present invention that is installed on a user's electronic computing device (e.g. smartphone, tablet, etc.) may also emit a series of reminders if a dose is not registered by the preprogrammed time. Additionally, a software application of the present invention that is running, e.g. on a system server, may send a reminder if a dose is not recorded, by the pre-programmed time, or the wrong dose is taken.

Various embodiments of the present disclosure comprise a smart pill organizer device (or smart packaging), comprising a container with a sheet of replaceable dose units able to be filled by a robotic system, and tagged with a patient specific unique identifier. The bottom of the container comprises components of an electronic computing system that enable the device to communicate wirelessly with various remote and local (e.g. a patient's smartphone) to determine if a patient is taking their medication on time; to send reminders to take a dose; to alert the patient and third party computers when doses are missed; and to automatedly order prescription refills as needed.

In an embodiment, the smart pill organizer device further comprises: a printed circuit board; electromagnetic sensors, e.g. beneath each dose unit able to detect when a dose is taken; a wireless transmitter; a battery; a memory; a microprocessor; and a speaker-buzzer.

In an embodiment, the smart pill organizer device further comprises: a) a lid covering the top surface of the device, which may comprise a graphical display and/or a camera; b) a plurality of removable dose units, each unit filled with one or more medications, and sealed closed on the top with: a paper or foil attached to the four edges (e.g. via cold press techniques well known in the art), or a cap that can be popped on/off, or a pivotable cap that is hinged at one edge to keep the lid attached; c) a removable carrier tray, with a plurality of holes that are sized to hold the dose units in a manner that the top of the dose unit is above the tray and the bottom of the dose unit is beneath the tray and the tray resides on top of the container; and d) a bottom unit comprising: a container with a plurality of receptacles of concave cavities for holding the removable dose units; an exterior case sized to fit the tray; and a button that is depressed by a user when a new carrier tray has been placed into the exterior case of the smart pill organizer, and/or to reorder a new tray of dose units (i.e. a prescription refill).

The smart pill organizer device can be a standalone product that communicates to a patient when to take a dose (e.g. via a speaker and/or display on the device). In a preferred embodiment, the smart pill organizer device is part of a networked system of electronic computing devices (e.g. patient's smartphone with a mobile application, pharmacy computer, doctor's office computer, and remote servers with patient records on a database). The networked system communicates with the smart pillbox device via a wireless network, which may include a voice activated hub (e.g. Amazon Echo®, Google® Assistant, or the like) that is linked to the pillbox device.

The various embodiments of the present disclosure further comprise; methods of automatedly tagging the dose units with a patient's unique identifier; manufacturing the dose units as a tagged sheet in a robotic filler, and cutting the sheet into individual units; non-transitory computer readable storage media comprising instructions for a processor (e.g. patient's mobile application; and system server software); methods of automated refilling and shipping trays of prescriptions; the networked computer system for patient monitoring and communication; etc.

In an embodiment, the pillbox may further comprise: at least one electrical and/or mechanical component for verifying the identity of a user before unlocking the pillbox container, comprising one or more of: a fingerprint scanner, a passcode reader, a two-factor authentication reader, a voice recognition device.

In an embodiment, the smart pill organizer device further comprises a lid, and comprises a touchscreen and a camera that are embedded into the lid or into other parts of the device, e.g. the removable carrier tray or the bottom unit, and enabled for conducting wireless communications with a remote healthcare provider or a caretaker.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

The accompanying drawings, which are incorporated in and constitute a part of this specification, illustrate certain aspects of the instant invention and, together with the description, serve to explain, without limitation, the principles of the invention.

FIG. 1 is an illustration of an exploded view of the smart pill organizer of the present invention.

FIG. 2A is a top perspective view of a dose unit with a paper or silicone cap seal.

FIG. 2B is a side view of one dose unit illustrating the ridge lines for the tray that the dose unit fits into.

FIG. 2C is a top perspective view of a dose unit with a pivotable cap in an open position.

FIG. 2D is a top perspective view of a dose unit illustrating text on the cap listing the medications stored within and amounts.

FIG. 3 is an illustration of an exemplary device of the present invention with 31 days of medication.

FIG. 4 is an illustration of an exemplary device of the present invention with 7 days of medication.

FIG. 5 is a top plan view of the smart pill organizer comprising a LED and sensor beneath each dose unit and connected to an electronic with wireless computing components to transmit data between the smart pill organizer, and the user's and/or caregiver's and/or pharmacy electronic computing device, and/or a remote server.

FIG. 6 is a side view of the device with a LED, and a magnetic or contact sensor beneath each dose unit, and connected to an underlying printed circuit board, to detect when a dose is taken.

FIG. 7 is a side view of the device with a sensor beneath each dose unit

FIG. 8 is a side view of the device with a sensor on the side of the tray.

FIG. 9A is an exemplification of using a light emitting diode (LED) under each dose unit to detect when a dose is taken.

FIG. 9B illustrates using a magnet in the lid with a reed switch to detect when the device is opened/closed.

FIG. 10 is a graph showing how LED light scattering is used to detect the number of pills in a container.

FIG. 11 illustrates using one or more load cells beneath the dose units and connected to the underlying printed circuit board to detect when a dose is taken.

FIG. 12 is a block diagram illustrating the primary hardware and software components of an exemplary smart pill organizer device.

FIG. 13A is an illustration of a networked system comprising a plurality of smart pill organizer devices communicating via a wireless network with a dose management system of the present invention, and third-party computers (e.g. caregiver, doctor, pharmacy, etc.).

FIG. 13B is another illustration of a networked, wireless system comprising a hub communicating with the device 90 and a central web application on remote servers.

FIG. 13C is another illustration of a networked, wireless system comprising a voice hub with API, connected to a pharmacy system, and remote patient database.

FIG. 13D is another illustration of a networked, wireless system comprising a voice hub with API, connected to a pharmacy system, patient smartphone, and a remote patient database.

FIGS. 14-27 are a series of flowchart of steps using the smart pillbox with the networked system of FIG. 13A.

FIG. 14 is a UML diagram showing the steps in a dose sheet sensor event synchronization.

FIG. 15 is a flowchart illustrating linking a dose sheet sensor to an organization (e.g. pharmacy, doctor's office, caregiver, dose management system) to be able for the organization to remotely monitor dose adherence, refill requests, etc. using the networked system of FIG. 13A-13D.

FIG. 16 provides a list of exemplary steps on how to wirelessly link the sensor to the smart pillbox and to the networked system of FIG. 15, and the organization that the new dose units are sent (e.g. pharmacy, direct mail delivery to patient, etc.).

FIG. 17 is a flowchart illustrating how device in an organization wirelessly communicates with a patient host.

FIG. 18 is a flowchart of computer steps disclosing how the system detects using a Bluetooth whether the user has taken their dose on time or not.

FIG. 19 is a flowchart of steps of the patient taking a video or photo of remaining pills and weight on scale and transmitting to remote server to prove dose taken, and order refill.

FIG. 20 is a flowchart illustrating a pharmacy filling a medication refill, sending to a patient with a unique identifier in the dose sheet, tray, etc.

FIG. 21 is another flowchart of steps of a user setting up the dose schedule from their smartphone, which has an alert/alarm when dose missed, and device monitoring when a patient takes a dose and communicating with a timestamp to a remote server.

FIG. 22 is a flowchart of computer system steps when the system is not able to connect with the smart device (e.g. Bluetooth off).

FIG. 23 is a flowchart of system steps monitoring when patient misses a dose, and alerting the patient smartphone and third-party computer via text when this occurs.

FIG. 24 is a flowchart of system steps on how a remote server (e.g. third party, organization, etc.) keeps connected to device using a key every 10 seconds.

### DETAILED DESCRIPTION OF EXEMPLARY EMBODIMENTS

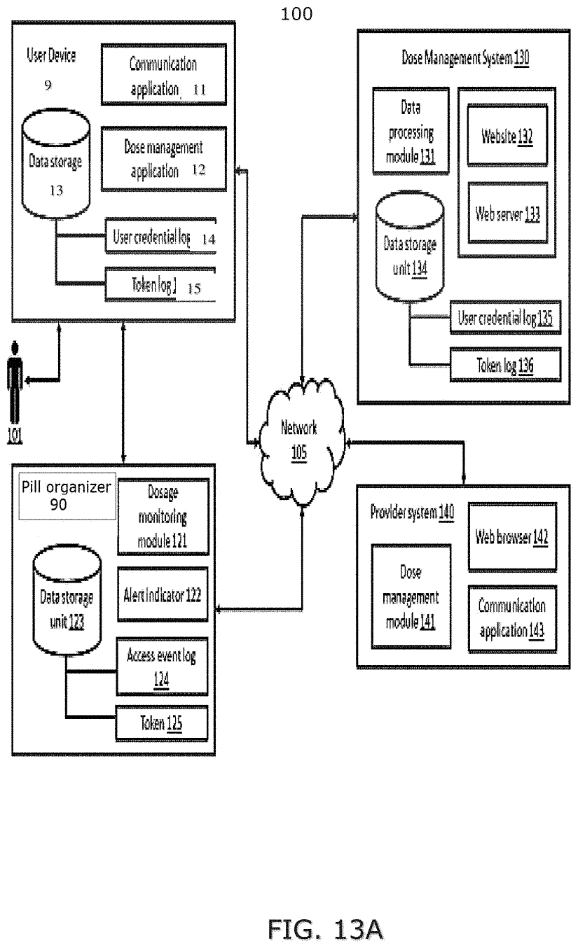

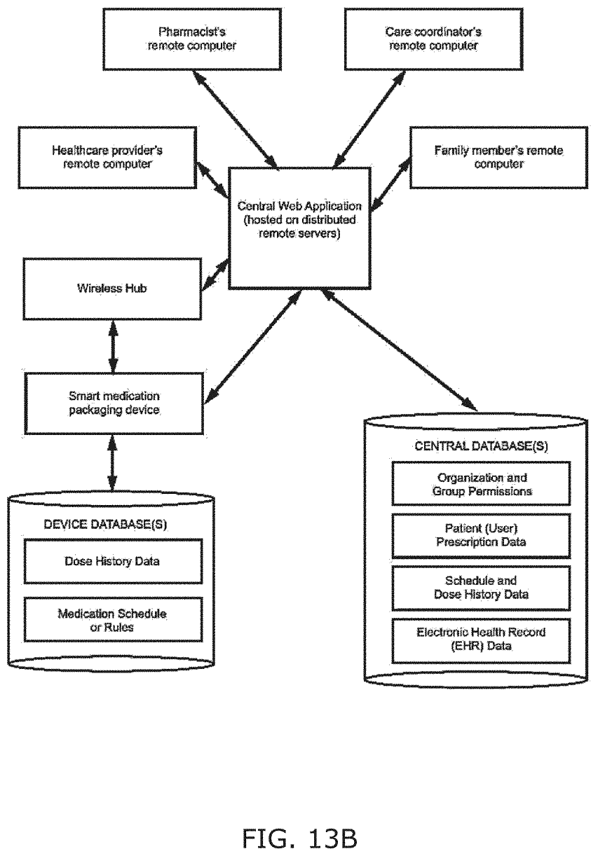

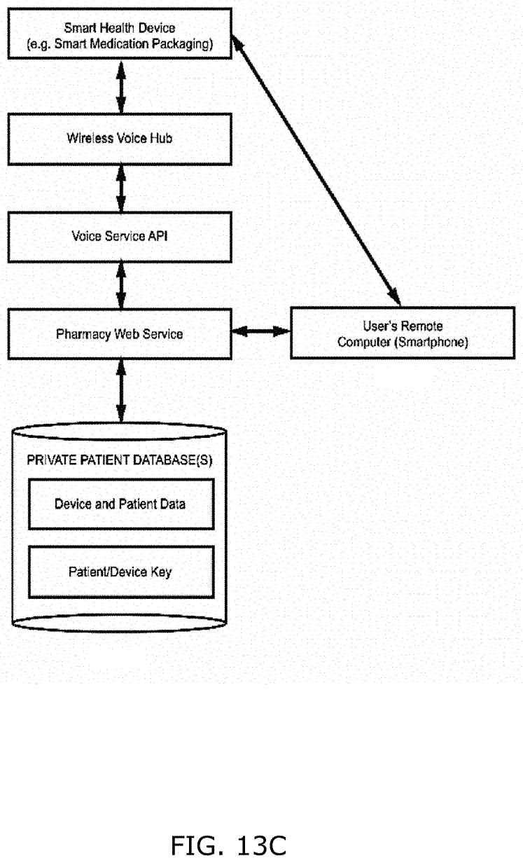

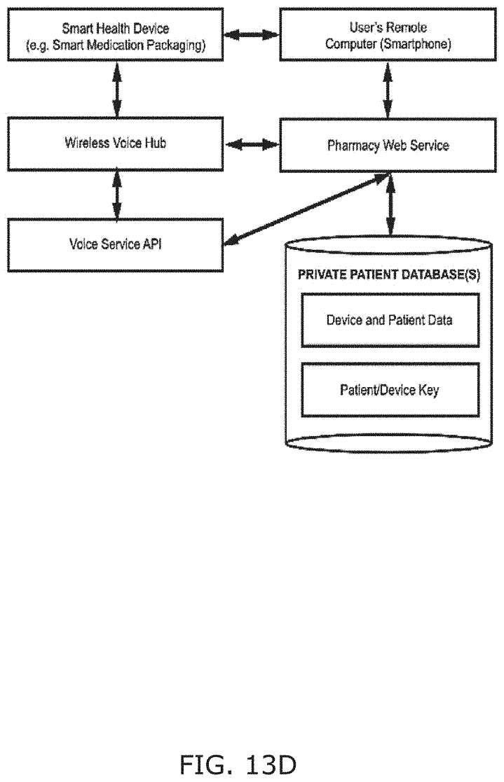

Smart device: the pill organizer device 90 of the present invention is a smart, wireless device that is able to connect to remote computers and a mobile application of the present invention to transmit and receive information about whether and when a patient has taken a dose of medication, per the networked system of FIG. 13A.

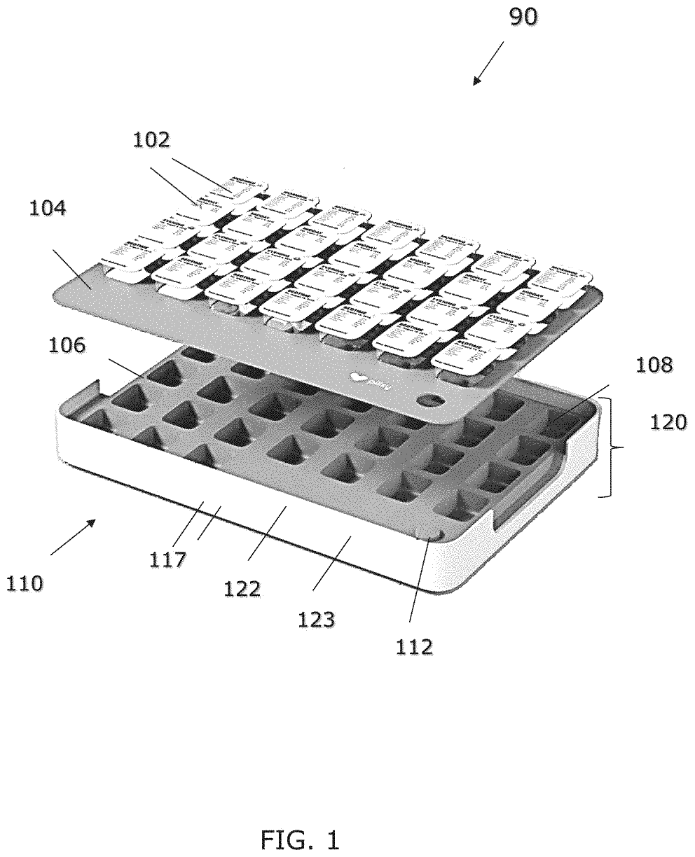

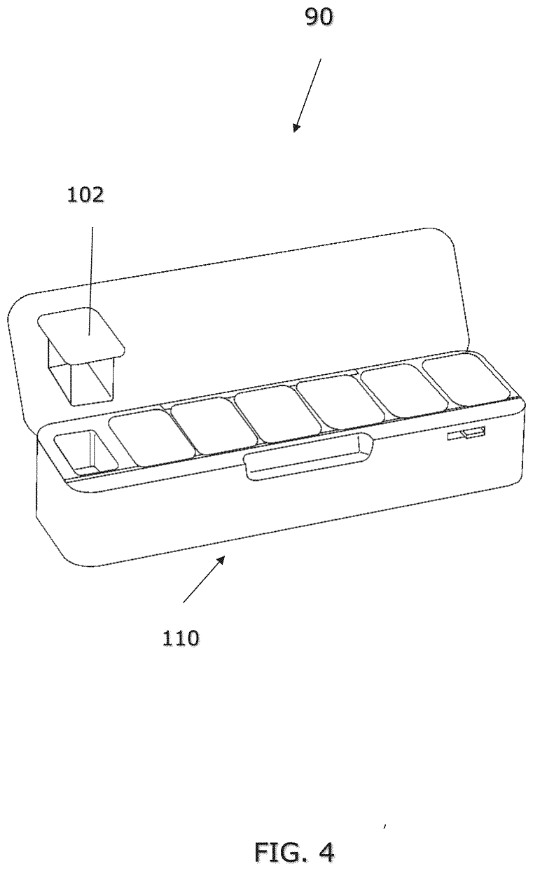

In an embodiment, as illustrated in FIG. 1, the smart pill organizer device 90 comprises the hardware components, from top to bottom, of: a lid (optional-not shown) covering the device 90 top surface; a plurality of dose units 102; tray 104; and a bottom organizer 120. The bottom organizer 120 further comprises: container 106 with a plurality of top holes sized to fit the dose units 102 and upon which tray 104 rests; button 112 positioned on the top surface of container 106; an electrical circuit within container 106 comprising a wireless computing circuit/chip 123 within or connected to a printed circuit board (PCB) 122, and connected to one or more sensors 124 housed within the organizer's interior to detect when a dose is taken. Furthermore, container 106 rests within a solid exterior case or housing 110. In an exemplary embodiment, the tray 104, container 106, and housing 110 are substantially square to rectangular shaped, e.g. to fit a 30-31 day medication supply. In another embodiment, exemplified in FIG. 4, the housing 110 is substantially a thin rectangular shape.

### Removable Dose Units 102

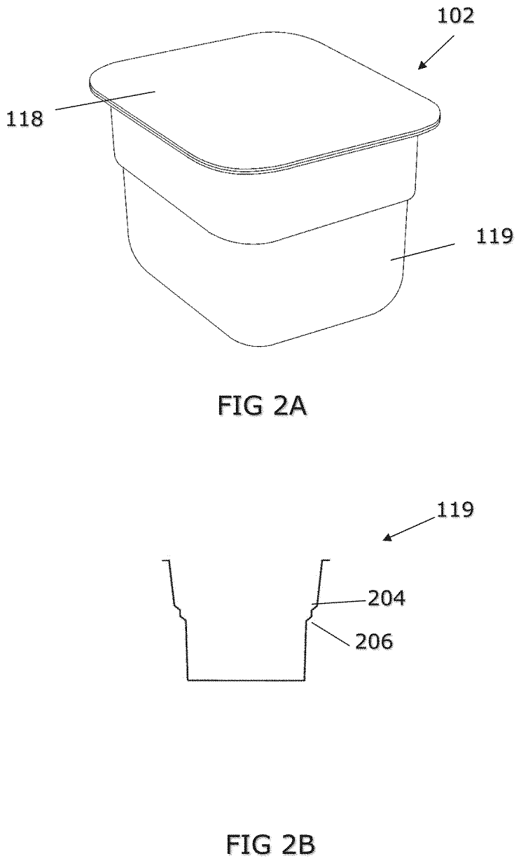

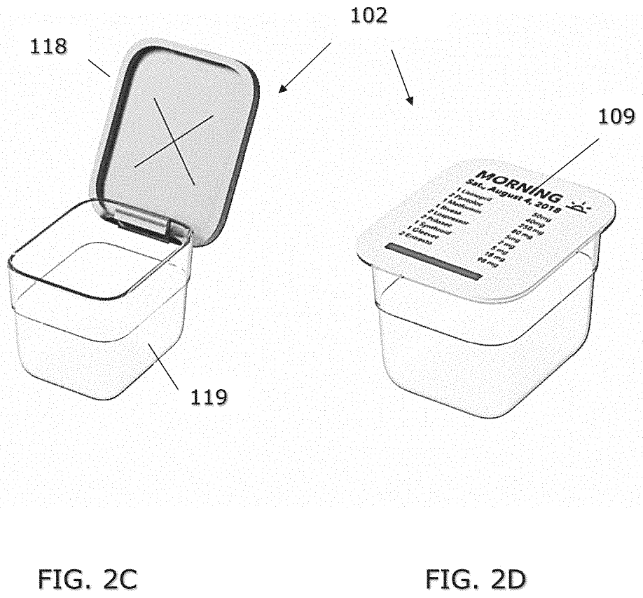

In the plurality of dose units 102 comprising a lid 118 covering a bottom container 119, each unit 120 is filled with one or more medications, and sealed closed. As illustrated in FIGS. 2A-2D, the seal may comprise either: a paper or foil attached to the four edges, or a cap 118 (e.g. silicone or the like) that can be popped on/off (FIG. 2A); or a pivotable cap that is hinged at one edge to keep the lid attached (FIG. 2C). The seal top surface may further comprise printed text 109 indicating the medication and dose within the unit 102 (and optionally a patient's name and when to take the dose) (FIG. 2D).

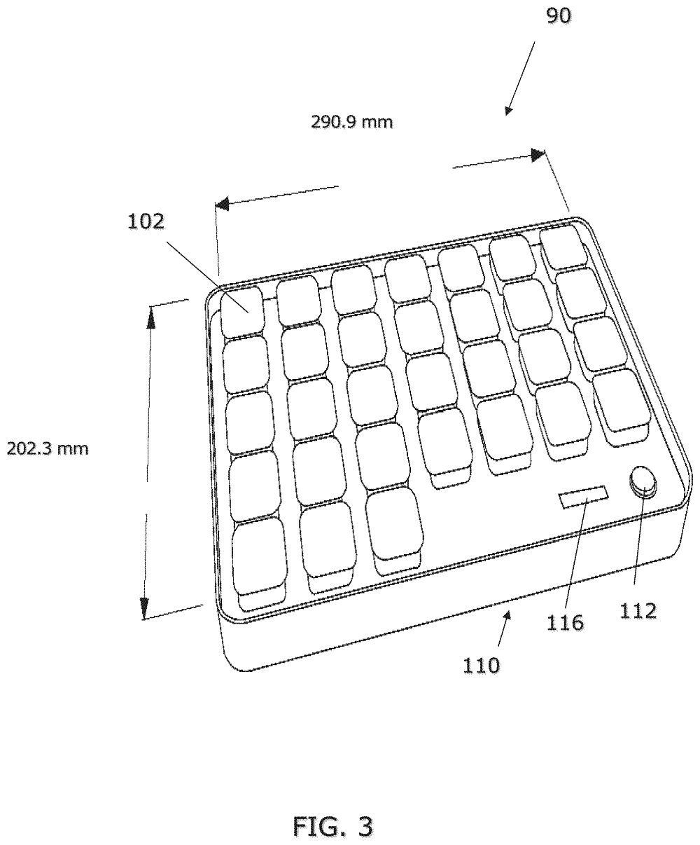

In one exemplary embodiment, device 90 includes a total of at least 31 dose units 102 fitted into 31 receptacles 108, and that are arranged in a grid of 7 by 4 or 5 dose units (see FIG. 3). Other configurations are envisioned within the scope of the present invention by the carrier tray 104 being altered to fit the number of does units 102, receptables 108, and their arrangement in rows (e.g. one row of 7 doses for a one week supply—e.g. see FIG. 4).

Cover (Aftermarket Version): To enable a version of the smart pill organizer that can be useful absent a pharmacy-enabled workflow, an optional cover may be placed on the dose pods to keep the pills inside of them. This mechanism may be used in lieu of a paper or plastic label that is affixed to the dose unit. In an ideal embodiment this material is silicone. In another embodiment the cover may have scores in the top, enabling pieces of the cover to be temporarily separated to place one or more pills into the dose unit without removing the cover.

Dimensions: The dimension range of the dose unit 102 is important, as there is a relatively narrow range that must be met to ensure that the dose units are large enough to hold a sufficient quantity of medications, while also forming a grid that is not too large to be filled by a robotic filling system, such as the Synmed™ system, or another robotic filling system made by Omnicell™. Exemplary ranges of dimensions of the dose unit 102 are: 1.4 inches wide×1.1 inches tall×1 inch deep.

In an exemplary embodiment, dose unit 102 has at least 1 tapered location on the exterior sidewall of the dose unit, and preferably two, the first tapered region catching the carrier tray holder (FIG. 2B, 204), and the second tapered region catching and resting on the dose unit cavity (FIG. 2B, 206). This mechanism enables the dose units to rest securely in the carrier tray 104 while having predictable fit and contact into the dose unit holder.

In an alternative embodiment, the bottom of the dose unit 102 may rest on the bottom of the receptacles 108, or on a protrusion pointing upward from the bottom of the receptacle 108, e.g. atop a sensor 124 to detect movement or change in weight of the dose unit, thus indicating a dose being taken.

Material: The material of the dose unit lid 118 and/or bottom container 119 is ideally a food grade or medication-grade plastic material, such as a polypropylene. In an ideal embodiment the material may be tinted amber color such as to prevent the transmission of light that could degrade the medication compounds inside the container. The material may also be transparent or an opaque color, such as opaque white.

Dose Unit Sheet, and Separation of Sheet: The dose units may originally be produced in a singular plastic sheet, with individual dose units being separated by perforated or scored marks between them. Such perforations or scores may be made by the mold or by a secondary process that is performed after the production of the dose unit tray 104. In an alternative embodiment the dose units may be held together by plastic rods that can be snapped off of the dose unit.

The dose units 120 may be separated either during or after filling. In either case, the dose units are likely to be placed in a template holder during the filling process. A printed label (e.g. medication, dose, patient, etc.) may be applied to a sheet of dose units after the dose units are filled with their respective medications. This printed label is likely to have an adhesive backing on one side. The other side of the label sheet is likely to accept a printable coating. In an ideal embodiment the label sheet has at least one dimension that is or equal to a standardized printing dimension, such as 8.5″×11″ or 11″×17″, such that the label can be easily trimmed or folded after printing.

### Tray 104

The removable carrier tray 104 is shaped to fit the top of container 106 (e.g. tray 104 is thin rectangular shaped in the exemplified embodiment of FIG. 1), and comprises a plurality of holes 103 that are sized and shaped to hold the dose units 102 in a manner that the top the dose unit is above the tray 104 and the bottom is beneath the tray 104.

The carrier tray 104 is made from a thin material, ideally between 0-20 mm in thickness, and most ideally between 1-10 mm in thickness. The carrier tray can be made of any rigid or semi-rigid material, including plastic, cardboard or metal. In an ideal embodiment the carrier tray is made of an injection molded or blow-molded piece of continuous plastic. The carrier tray can hold a plurality of dose units, most ideally in a multiple of 7, 28, 30 or 31.

### Organizer Bottom Unit 110

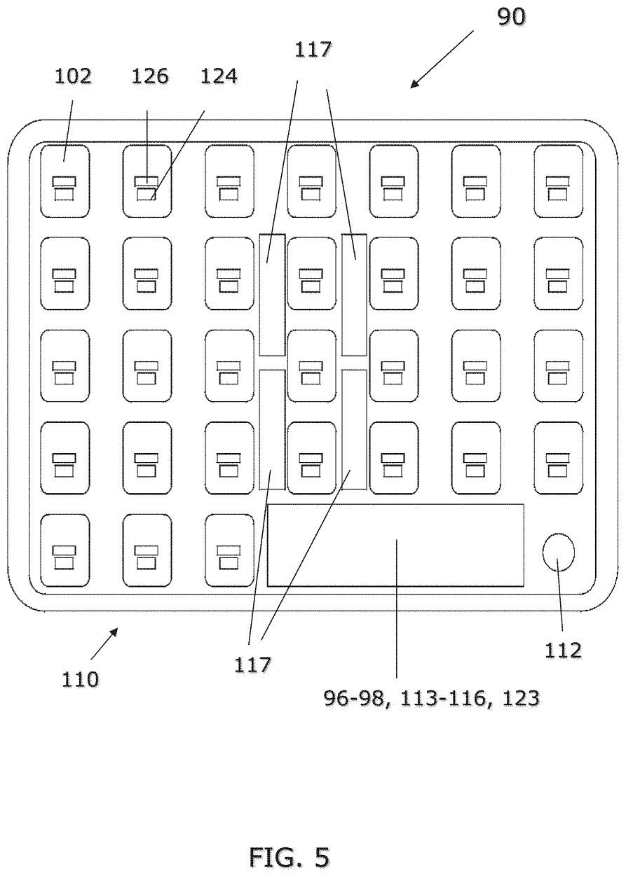

The smart pillbox organizer bottom unit 110 is comprised of: a container 106 comprising a plurality of receptacles or concave cavities 108, for insertion of the removable dose units 102; an exterior case 110 sized to fit tray 106 (and optionally with a lid—e.g. see FIG. 5); and a button 112 that is depressed by the user when a new carrier tray 106 has been placed into the exterior case 110 of the smart pill organizer.

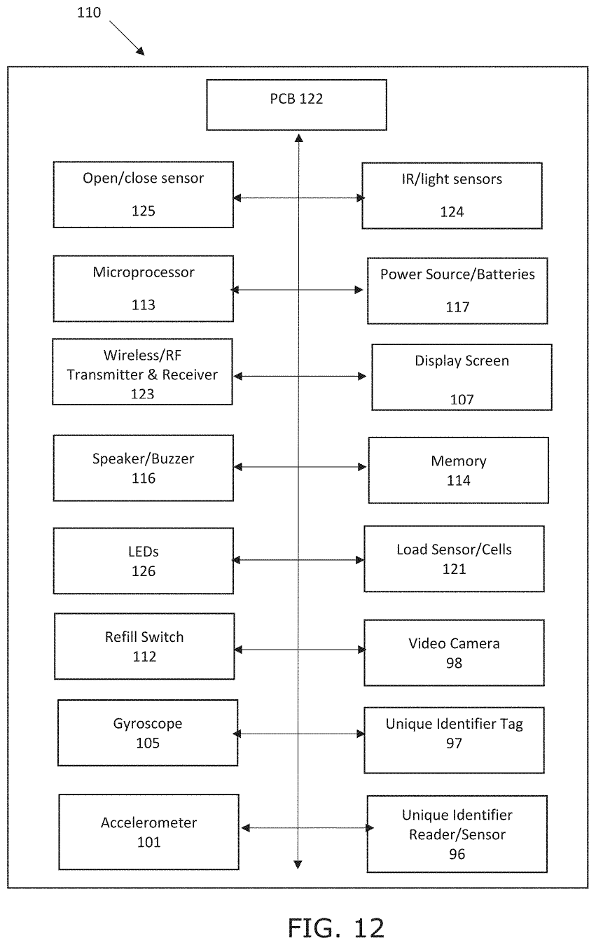

Organizer 110 further comprises the following electrical components, as illustrated in FIGS. 1- 12: a microcontroller 115 comprising a printed circuit board 122 housing a memory 114, at least one processor/microprocessor 113, a wireless transceiver-radio 123 (Bluetooth®, cellular, WiFi chip, non-infrared, etc.), and connected to at least one battery 117 (e.g. replaceable coin-shaped, atop or above the PCB, or 4 AA batteries); and (optional) a speaker/buzzer 116 able to emit audio messages to the user (e.g. reminder to take dose). The wireless radio 123 can further connect to Bluetooth enabled remote or local patient health monitoring devices (e.g. a blood pressure cuff), which transmits the information to the network. The wireless cellular radio 123 may further function as a wireless Hub for the monitoring devices to wirelessly transmit the data to a remote server/computer (e.g. doctor's patient file database in a cloud). The speaker 116 can also be used for voice-initiated medication refill, that can only be ordered after verifying a patient's identification. As illustrated in FIG. 12, the printed circuit board 122 connects an array of sensors 124, the battery power source 117, a microprocessor 113, the wireless radio or module 123, an antenna, a buzzer, memory 114, and (optionally) a cellular modem. The printed circuit board may also be in communication to a plug-in or external power source (e.g. USB cable), a speaker 116, and a color touch-screen display 107 (e.g. on a user's electronic computing device, or on tray 104, or on container 110).

In an embodiment, an electric circuit is printed or overlaid over each dose, at least a portion of the circuit being designed to broken when the pills interior to the dose unit are removed from the dose unit, and where this disruption in the circuit or antenna is detected, at least in part, by the microcontroller. Additionally, or in another embodiment, the circuit is printed to have different combinations of resistance or capacitance between doses, the removal of a dose being measured by detecting the change in the circuit's resistance or capacitance.

In one exemplary embodiment, organizer 110 further comprises: a printed circuit board 122 or flexible circuit that connects components: a first series of sensors 124, one beneath each dose unit 102, each sensor is activated and deactivated by removing a dose unit 102 from the container 106; a second sensor 125, which is activated by the opening and closing of the lid; an accelerometer, which detects motion of the device 90; a gyroscope 105, which detects orientation towards the gravity vector; a button 112, which can be pressed by the users upon the device refill to reset the sensors and PCB; a wireless radio(s) 123, capable of transmitting a signal to a cellular network and/or to a nearby WiFi router or remote computer; an energy storage device, e.g. a replaceable battery 117, ideally of lithium or alkaline chemistry; a local computer memory 114; and at least one processor/microprocessor 113.

Wireless radio: While the wireless radio may be of any spectrum, an ideal embodiment it is a low-power cellular radio, such as a NbIoT or LTE-M.

In an alternative embodiment, it may be a short-range wireless radio, such as a radio that operates at the 802.11 standard, or as originally defined by the IEEE 802.15.1 standard. In another ideal embodiment the frequency of the radio is between 2400 and 2489 MHz or at the 2.4 GHz frequency range.

Microcontroller and Operating Modes: The microcontroller can be programmed to include a variable for the mode that the smart pill organizer 90 is operating in, the mode being tied to the configuration of the dose units. For example, a weekly mode could be set to expect 7×4 or 7×3 doses, whereby a monthly mode could be set to include 28, 30 or 31 doses, depending upon the month. In this sense, the smart pill organizer is designed to store information on the device including a) whether it is operating on a weekly or monthly basis b) the number of doses per day c) the starting day or date associated with at least one of the dose unit receptacles d) a time of day associated with each mode.

Color Touchscreen Display: Optionally, the smart pill organizer may include a color touchscreen display 107, the display showing per Table 1:

TABLE 1
The next time that a dose is scheduled, or remaining time until the
next dose.
A warning if a double-dose is about to occur.
Confirmation that the correct dose was removed.
A list of medications
A picture of what a specific medication is supposed to look like.
General health tips, such as those that are related to general wellness
(e.g. blood pressure facts or weight management tip) or management of a
specific medication condition (e.g. hypertension or diabetes).

Video Camera: Optionally, the smart pill organizer may also include a video camera 98 embedded in container 110 (e.g. lid) or connected to the PCB 122 (e.g. via a USB cable to a smartphone 9 of FIG. 13A). Video may be streamed in near-real time, such as with a doctor's visit, or asynchronously, such as to record a video of the patient putting pills into their mouth. In an embodiment, a touchscreen display and a camera are embedded into the lid of the device, such as by using a low-cost Android or Raspberry Pi-based device; and it would be enabled for telehealth and other content to be played directly on the device.

Locking Mechanism and Refills: The smart pillbox may further comprise a lock on the exterior container with the lid 110 that requires verifying the identity of the user before it can be opened and the dose accessed or a refill ordered. By way of non-limiting examples, the container with the lid 110 may further comprise one or more electrical and/or mechanical components comprising: a fingerprint scanner, a passcode, a two-factor authentication, or a voice recognition. Preference is for a biometric verification that is performed locally on the device 90 (e.g. fingerprint scan or voice recognition). In an embodiment, the pillbox 90 is unlocked in response to a combination of a user action and an identification verification. User action comprises, e.g.: pressing a button on the container (e.g. button 112), touching a fingerprint scanner, or issuing a voice command, or touching a capacitance sensor on the pillbox itself. In another embodiment, the user identification must be verified before a refill is ordered, e.g. via a voice-initiated refill that can only be ordered after verifying identification using one of the listed methods.

### Device Sensors

### RFID or Other Unique Identifier

An RFID tag, NFC tag, Bluetooth beacon or other unique identifier 97 may be attached to each dose sheet or carrier tray 104. This unique identifier 97 may be read by an internal reader/sensor 96 within or accessible to the smart pill organizer when a new set of dose units 102 is placed into the smart pill organizer 90. When this is detected, the smart pill organizer microprocessor may: a) reset the dosing schedule; and/or, b) lookup the corresponding dose units and schedule that was included. This information is stored on the memory of the organizer 90, and/or is wirelessly accessible via the network connecting with one or more of: the user's, and/or caregiver's, and/or patient record on a pharmacy computer or on a remote server of the present invention (e.g. see FIG. 13A). The RFID tag is also able to detect when a dose is missed, an incorrect dose it taken, and trigger an alarm emitted from a patient electronic computing device.

In an embodiment, one or more unique wireless tags 97 are attached to the sheet of dose units, each tag having a unique identifier and corresponding to a unique dose unit, the unique identifier being correlated to a unique combination of medications or pills, a day, and a scheduled dose time window, whether in local storage on the device or in the cloud-based dose management system, and wherein the smart pill organizer device may also include a wireless reader/sensor 96 for such tags. For example, an NFC or RFID tag 97 covers each dose unit so the system can detect removal of each dose unit.

In another embodiment, the wireless tag 97 has a unique ID that correlates to a unique medication or combination of medications and a unique day, dose time, and dose window either in local storage on the device or in the cloud-based dose management system.

### Electromagnetic Material for Sensing Purposes

In order to facilitate detection of an electromagnetic sensor, each dose unit 102 may be doped with, or be attached to an additional material with readily detectable electromagnetic properties. For example, in one embodiment a portion of the plastic material could be doped with a magnetic or conductive material, to result in a magnetic or electromagnetic piece of plastic. In another embodiment a magnet or metallic circle, fragment or strip could be attached to the bottom of the dose unit, including by using an adhesive. Such a material would not need to be large but would ideally be large enough to manipulate with a finger, (e.g. between 1 and 90 square millimeters). In an alternative embodiment an RFID or NFC tag 97 or sticker could be placed on the bottle of each dose unit—this would have the additional benefit of detecting the removable of an individually-labeled dose.

### Sensing Mechanism

Prior art wireless pill organizers disclose the use of mechanical switches to measure the removal of dose units. In contrast, the present invention teaches the use of various electromagnetic sensors (see FIGS. 5-8). In one embodiment, a low-power hall effect (e.g. ultra-low power 1.65 V to 5.5V Hall Effect Switch DRV5032 from Texas Instruments™) can be used to measure removal of a dose. In another embodiment, a capacitance sensor can be used to measure removal of a dose. In either case, the electromagnetic sensor detects removal of the dose unit and/or any magnetic or conductive material that is attached to the dose unit 102.

FIG. 5 is a top plan view of the pill organizer 90 with a light sensor 124 beneath each dose unit 102 to detect the removal of the dose unit, or even the removal of individual pills. With this sensor design, a light 126 (e.g. from a light emitting diode) that is placed under each dose unit 102 pulses light at a known wavelength (e.g. UV). A light sensor 124 that is located under the same pod detects the scattering of the emitted light. With this method, the amount of light that is detected will be correlated to: a) the presence of the dose unit 102; and b) the presence of pills inside of the dose unit (if the dose unit material is at least partially transparent).

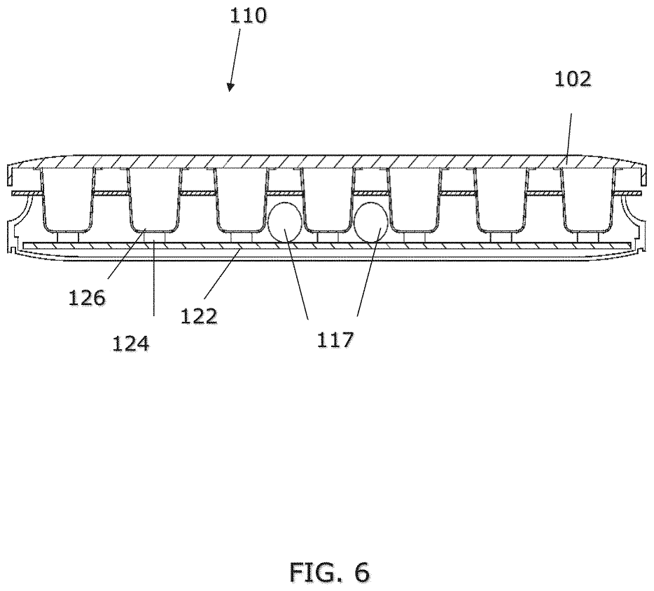

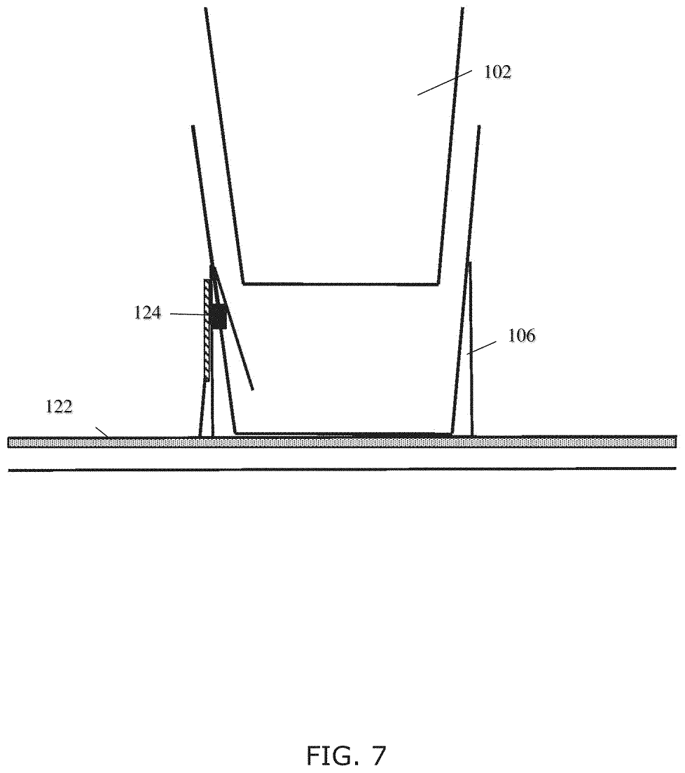

FIG. 6 is a side view pill organizer illustrating a printed circuit board (PCB) 122 positioned horizontally below the dose units 102. A magnet or metallic circuit or LED 126 resides in the bottom of the dose unit 102 atop a magnetic switch or sensor 124. When a dose unit 102 is removed, the sensor 124 detects the removal, and relays the information to the PCB 122.

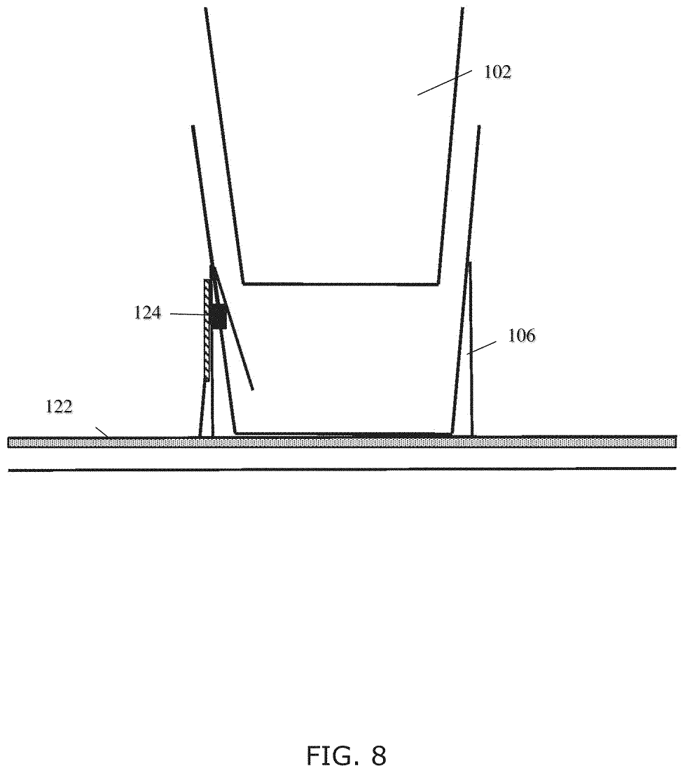

FIG. 7 is a side view of the device with another embodiment of one or more sensors 124 (e.g. a capacitance or magnetic switch sensor) beneath each dose unit 102.

FIG. 8 is a side view of the device with another embodiment of a sensor 124 (e.g. a capacitance or magnetic switch sensor) on a side wall of the tray 106 dose unit.

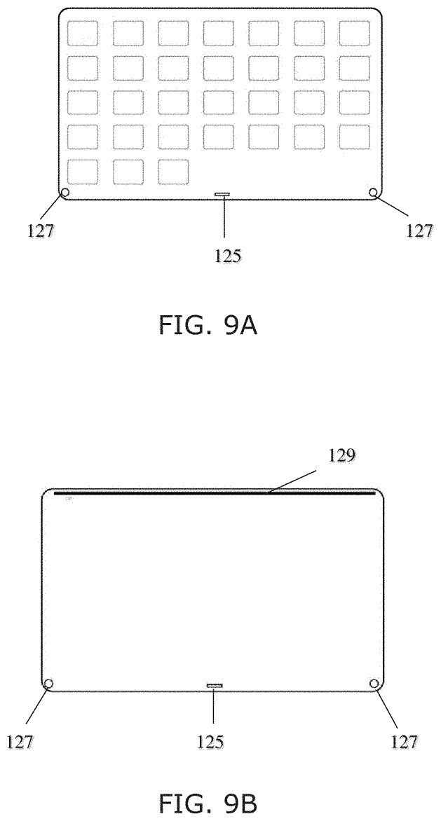

FIG. 9A is a top plain view of a dose container 90 with the lid off; and FIG. 9B is a top plan view with the container lid closed, and with the container further comprising: a magnet 127 to keep the lid closed, a reed switch 125 for detecting the lid is open/closed, and a hinge magnet 129.

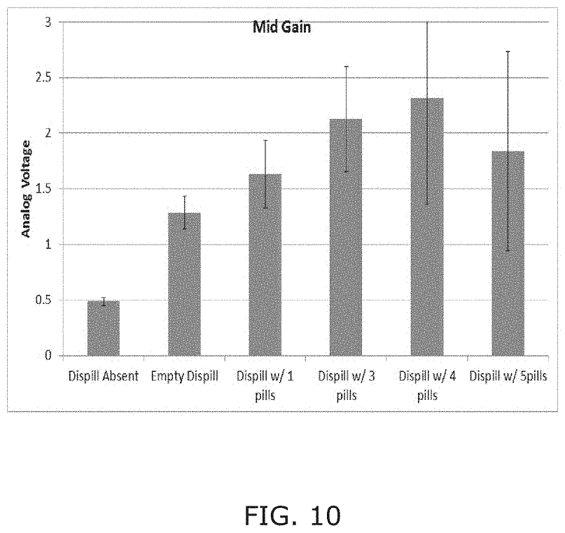

FIG. 10 is a graph that shows a correlation between number of pills in each dose unit 102 and light scattering (e.g. voltage) using this method. When a pill is removed from 5 pills to 4 pills in a unit 102, the voltage drops from about 2.25 volts to about 1.75 volts, indicating a pill has been removed.

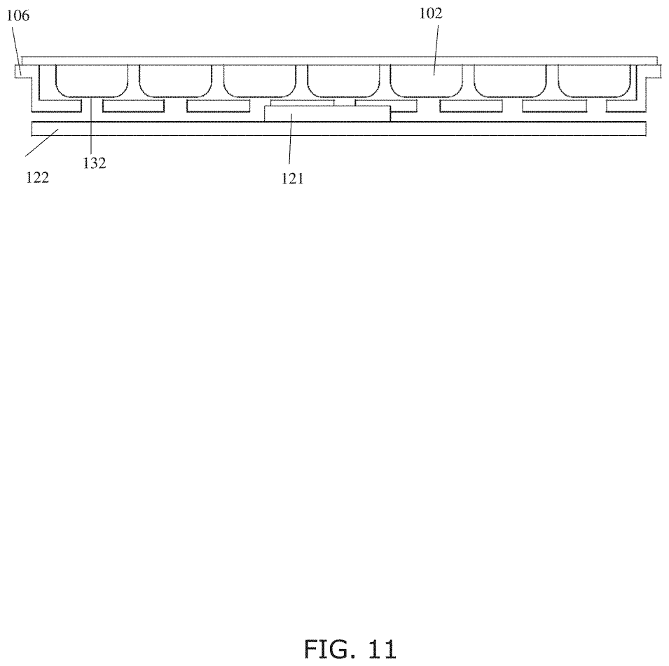

In FIG. 11, one or more load cells 121 is connected to a printed circuit board 122, and beneath the dose units 102 to detect when a dose is taken. The tray 106 may further comprise a plurality of holes in the bottom layer, versus individual sections for each dose unit 102. In another embodiment, the sensor 121 is the wireless tag reader 96 that is connected to the printed circuit board 122, and wherein the wireless tag 132 is also attached to each removable dose unit.

### Method of Filling the Dose Units

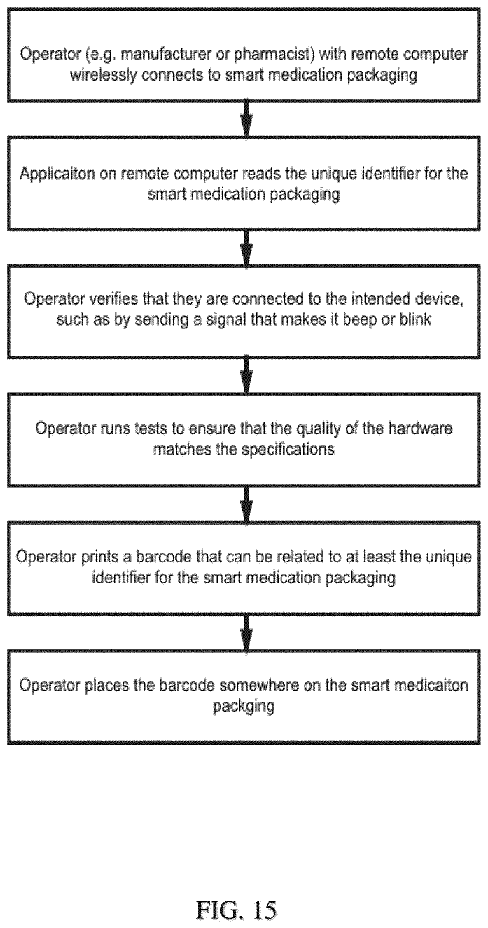

The dose units are robotically filled and placed into the box using the following method as listed in Table 2. In an embodiment, at least one sensor of FIGS. 5-8 is attached to the device 90 (e.g. the dose sheet, tray, etc.) to complete the steps of FIG. 15 that wirelessly links and tracks each new tray 104 of dose units with the networked system of FIGS. 13A-13D.

TABLE 2

| | |
|---|---|
| 1. | A dose unit sheet is placed into a concave filling template, which provides stability. The dose units |
| | sheets comprise the plurality of dose units 102, joined into the one sheet or formed by the robotic |
| | system by pressing a flat sheet. |
| 2. | The filling template and dose unit sheet are placed into the filling station within a robotic filling |
| | system, such as a Synmed ™ System. |
| 3. | The robotic filling system is programmed with the corresponding medication list and dose schedule, |
| | and automatedly fills the dose unit sheet with medications per receptacle 106. |
| 4. | The dose unit sheet and template are removed from the filling station. Optionally, an inspection of |
| | the filled dose unit sheet is performed. This can be a vision-based or weight-based inspection, where |
| | the expected value is compared to an observed value. If the dose unit sheet passes the test, then it is |
| | moved to a printing station. |
| 5. | A corresponding dose unit label sheet comprising a plurality of labels 119 (one per dose unit 102) |
| | is printed. Optionally, a barcode or other unique identifier 97 may be first scanned to lookup the |
| | corresponding patient and their medication list and dose schedule. This can be done using an |
| | adhesive-backed sheet, or by using a heat-based sealing system. The dose unit label sheet is affixed |
| | to the dose unit sheet. |
| 6. | The dose units 102 are separated. This can be pre-formed by using a templated cutting or separation |
| | device, or by a laser cutter. |
| 7. | The dose units 102 are removed and placed into a corresponding carrier tray 104; and it is placed |
| | into a box for shipping; and mailed or delivered to the corresponding patient. |
| 8. | The corresponding patient or their caregiver removes the carrier tray and places it into the smart |
| | pill organizer device 90 by resting the carrier tray 104 into the corresponding dose receptacle 106, |
| | 110. |
| 9. | The patient or caregiver may press a button 112, scan a barcode, or perform another action to |
| | indicate that the new carrier tray 104 has been placed into the smart pill organizer. |
| 10. | The smart pill organizer provides an audio or visual cue to indicate that a new smart pill organizer |
| | has been detected by the networked system (e.g. FIG. 13A, Dose Management System 130, |
| | Provider System 140). |

### Networked System

The smart pill organizer device 90 of the present invention may be used as a stand-alone device for a user to receive verbal messages for the device's speaker to remind them to take their medication on time. Or, preferably, the device 90 is used in conjunction with the networked system of FIGS. 13A-13D. The various embodiments of the present invention can be used in conjunction with the networked computer system in FIG. 13A with the dose management system (FIG. 1, 130) and the provider system (e.g. pharmacy 140); and the smart monitor device (FIG. 1, cap and bottle) is equivalent to the smart pill organizer device 90 of the present invention as disclosed in SYSTEM, METHODS, & DEVICE FOR MANAGING A PRODUCT, WIPO Patent Application WO/2019/006212 A1, Filed: Jun. 28, 2018 by LeBrun et al, and Published: Jan. 3, 2019. The entire disclosure of the above-identified patent application is hereby fully incorporated herein by reference in its entirety.

The smart pill organizer device 90 is able to be pre-programmed with scheduled doses. If a dose is taken within a pre-programmed dose interval, then the dose will be automatically recorded and a reminder will not occur. If the dose is not taken, then the organizer device will emit a reminder. Optionally, a mobile application of the present invention that is installed on a user's electronic computing device (e.g. smartphone, tablet, etc.) may also emit a series of reminders if a dose is not registered by the preprogrammed time. Additionally, a software application of the present invention that is running on a system server may send a reminder if a dose is not recorded by the pre-programmed time.

Messaging may also be delivered depending upon the last time the data from the smart pill organizer device has been synced to the internet. For example, if the last time the user connected to the internet was 48 hours ago, then the user may receive a message asking them to connect to the internet, where if they are currently connected to the internet then the message may remind them to take their medication.

An important part of this application is the connection of data. In the preferred embodiment a remote caregiver, such as a pharmacist, has access to the data through a clinical support platform, the clinical support platform providing: a) a way to view medication adherence data; and b) (preferably) a way to communicate with the patient (e.g. electronic messaging, email, etc.).

Automated Prescription Refill: In another embodiment, an activation of the switch or button (FIG. 1, 112) may initiate a refill request that is sent to the user's pharmacy. For example, if the user presses the switch twice within a predefined period of time (e.g. 1 second) a refill signal may be sent to the mobile app. This signal may then be relayed to servers, which are relayed to the user's pharmacy system. Alternatively, the timestamp of the button press may be recorded, with a filter running on the servers interpreting two clicks within a period of time (e.g. 1 second) as an indication to send a refill request to the user's pharmacy. Optionally, the user may receive a confirmation message via SMS or in their mobile app prior to sending the refill request to their pharmacy. Other methods of automated ordering of refills are listed in some of the flowcharts below.

### Dose Sensor and Organization Communication

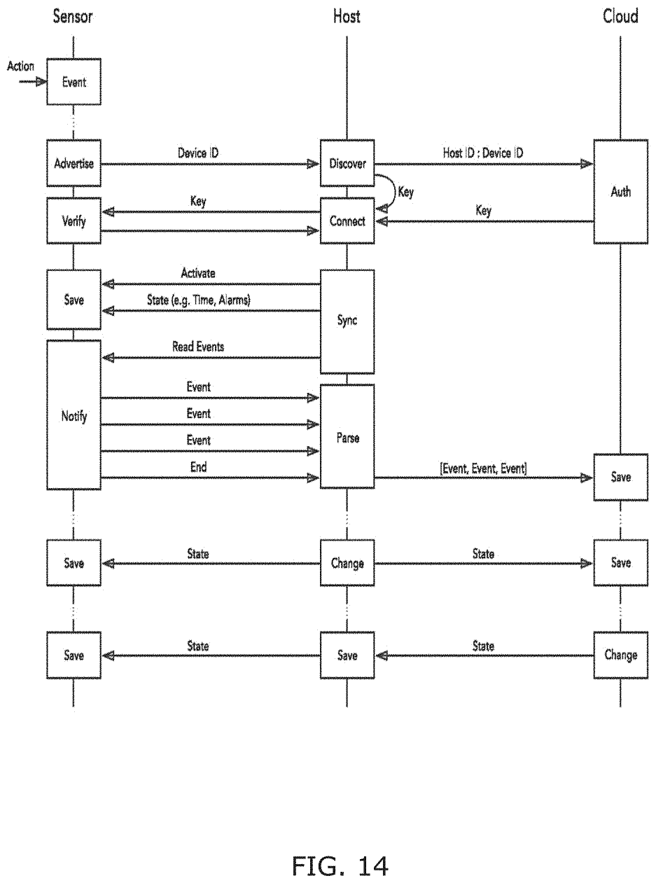

FIGS. 14-24 are various flowcharts of computer/user steps in utilizing the device 90 with the system 100 of the present invention.

FIG. 14 is a dose sensor event synchronization UML flowchart.

FIG. 15 is a flowchart of steps of a manufacturer of the dose units linking a unique identifier to a patient and the dose units, and then placing a barcode with the unique identifier encoded within it, onto the does units (e.g. dose sheet) before shipping.

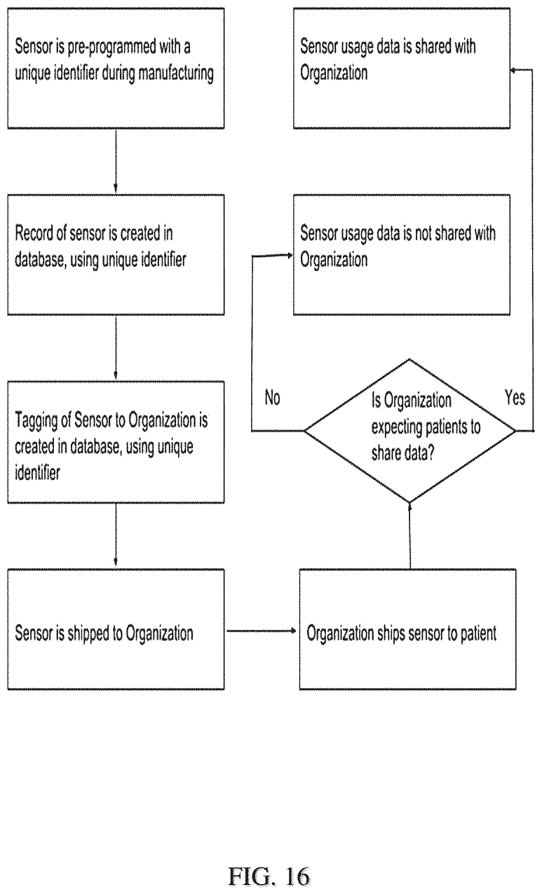

FIG. 16 provides a list of exemplary steps on how to wirelessly link the sensor of FIGS. 9-14 to the device 90 and to the networked system of FIG. 15, and the organization that the new dose units are sent (e.g. pharmacy, direct mail delivery to patient, etc.).

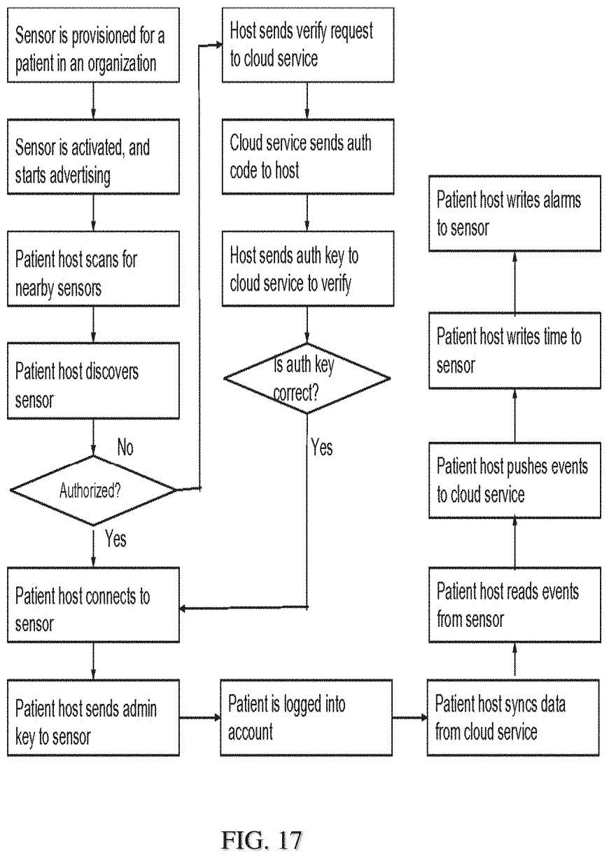

FIG. 17 is a flowchart illustrating how device 90 in an organization wirelessly communicates with a patient host.

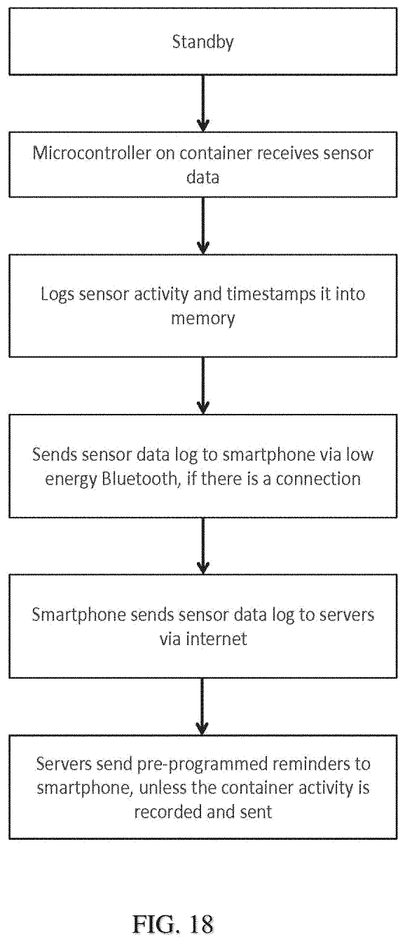

FIG. 18 is a flowchart of computer steps disclosing how the system detects using a Bluetooth whether the user has taken their dose on time or not.

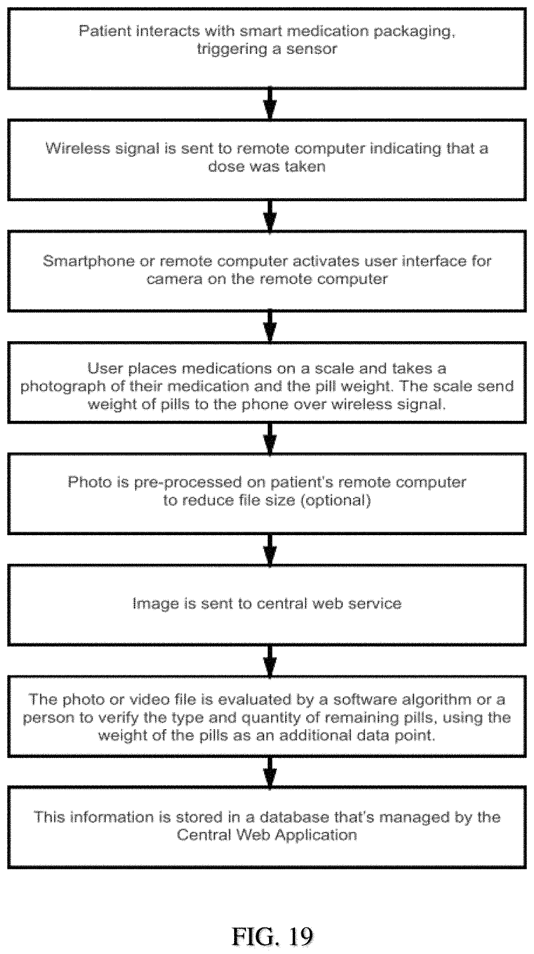

FIG. 19 is a flowchart of steps of the patient taking a video or photo of remaining pills and weight on scale and transmitting to remote server to prove dose taken, and order refill.

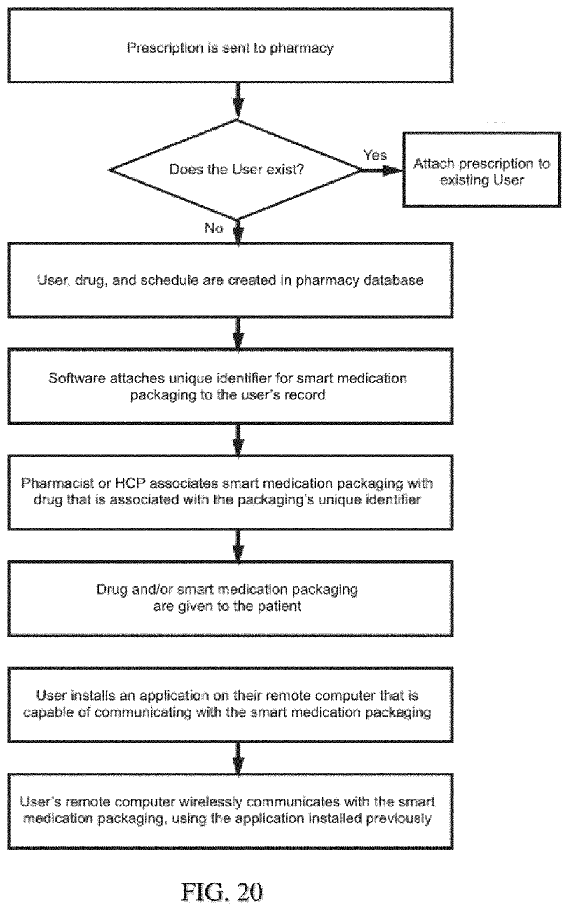

FIG. 20 is a flowchart illustrating a pharmacy filling a medication refill, sending to a patient with a unique identifier in the dose sheet, tray, etc. The patient then uses a mobile application of the present invention (e.g. non transitory computer readable storage media) to wirelessly communicate with the device 90 (e.g. receive electronic message for reminder to take dose).

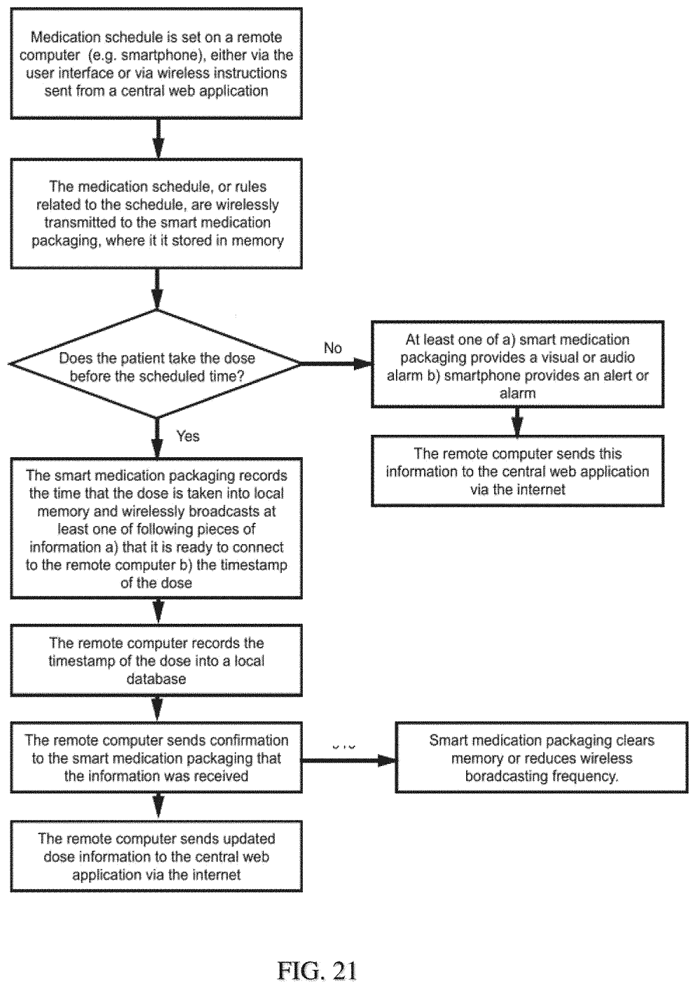

FIG. 21 is another flowchart of steps of a user setting up the dose schedule from their smartphone, which has an alert/alarm when dose missed, and device 90 monitoring when a patient takes a dose and communicating with a timestamp to a remote server.

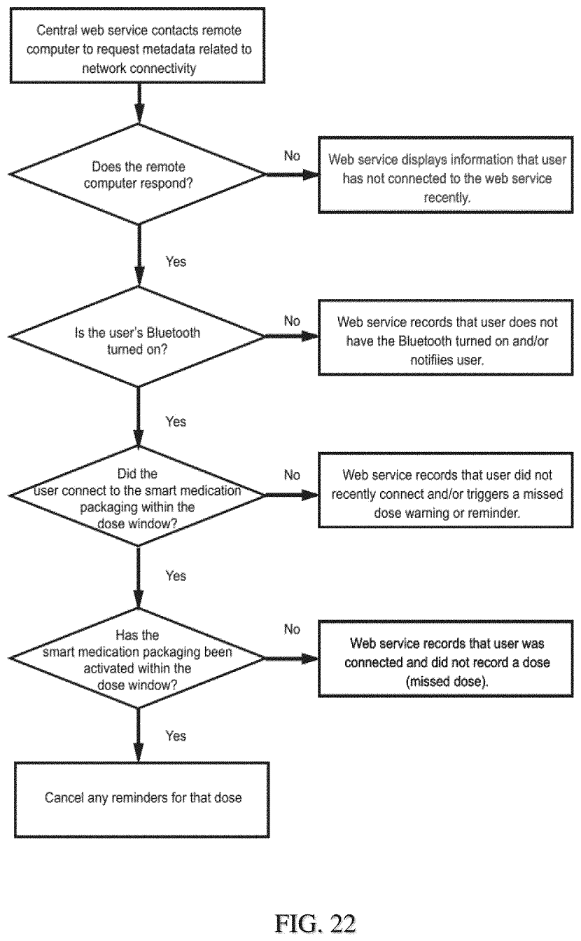

FIG. 22 is a flowchart of system steps when the system is not able to sync with the device to receive data on when or if the last dose was taken (e.g. Bluetooth is off) and when smart packaging does not detect that a dose is taken.

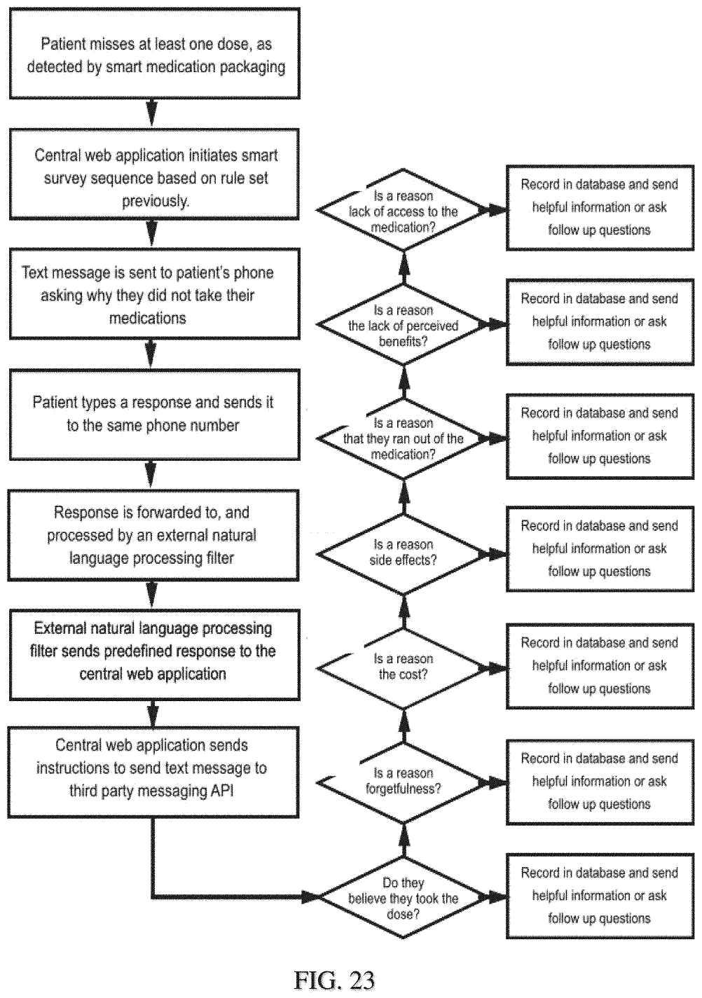

FIG. 23 is a flowchart of system steps monitoring when patient misses a dose, and alerting the patient smartphone and third-party computer via text when this occurs.

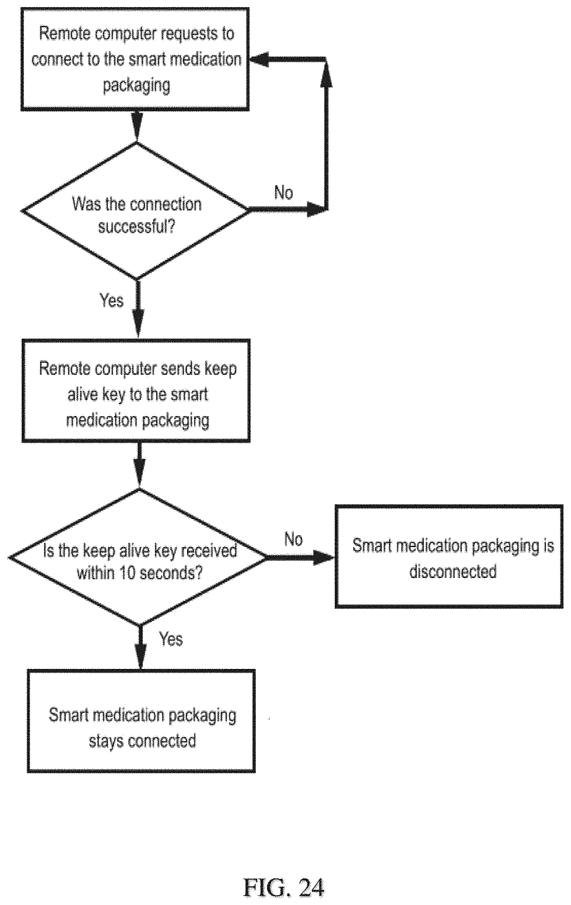

FIG. 24 is a flowchart of system steps on how a remote server (e.g. third party, organization, etc.) keeps connected to device 90 using a key every 10 seconds.

### CONCLUSION

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the inventions described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Or, the technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 5%), and use of the term “about” at the beginning of a string of values modifies each of the values (i.e., “about 1, 2 and 3” refers to about 1, about 2 and about 3). In a preferred embodiment, about refers to plus or minus up to 25% of the stated value. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

As used herein, the term “substantially” refers to approximately the same shape as stated.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments.

Trademarks: the product names used in this document are for identification purposes only; and are the property of their respective owners.

1. A smart pill organizer device, comprising a pillbox container holding a plurality of individual removeable dose units fillable by a robotic system, the pillbox container comprising from top to bottom:
a) a removable carrier tray, with a plurality of holes that are sized to hold a plurality of individual removeable dose units in a manner that a top of the dose unit is above the tray and a bottom of the dose unit is beneath the tray;
b) a bottom unit, comprising:
i. a bottom container comprising a plurality of top holes sized to fit the dose units when the carrier tray rests upon the bottom container;
ii. a button on the bottom container that is able to be depressed by a user to manually reorder a prescription refill, and/or to reset the device to restart dose adherence monitoring, and/or to initiate a call to a pharmacist or a medical provider;
iii. an exterior case sized to hold the bottom container;
c) the pillbox container further comprising:
i. at least one first sensor, positioned beneath each dose unit or one beneath all dose units, each sensor able to be activated and deactivated by removing one dose unit, or pills within the dose unit, from the pillbox container;
ii. a wireless radio capable of: transmitting a signal to a cellular network, and/or to a nearby WiFi router, and/or a remote computer, and/or a Bluetooth remote monitoring peripheral;
iii. a replaceable battery;
iv. a local computer memory;
v. at least one processor or a microprocessor;
vi. a printed circuit board connecting all electrical components within the pillbox container;
d) wherein the plurality of individual removable dose units are each filled with one or more medications, labelled, and sealed closed with: a paper or foil, or a cap that can be popped on/off, or a pivotable cap that is hinged at one edge to keep the lid attached;
c) e) wherein the smart pill organizer device is able to communicate with one or more electronic computing devices via a wireless network to track a patient's medication adherence, send alerts when doses are missed, and order refills automatedly and manually; and
d) f) wherein the plurality of individual removable dose units are derived from a sheet of dose units that are able to be filled by a robotic system, tagged with a patient specific unique identifier, and separated into said individual removable dose units.

2. (canceled)

3. The smart pill organizer device of claim 1, further comprising a lid, and comprising a touchscreen and a camera that are embedded into the lid or into the removable carrier tray or the bottom unit, and enabled for conducting wireless communications with a remote healthcare provider or a caretaker.

4. The smart pill organizer device of claim 3, further comprising one or more of: a second sensor, able to be activated by the opening and closing of the lid; an accelerometer able to detect a pillbox container motion; and a gyroscope able to detect orientation of the pillbox container towards a gravity vector.

5. The smart pill organizer device of claim 1, wherein said smart pill organizer device is able to wirelessly communicate with at least one remote computer or electronic computing device comprising: a pharmacy computer, a caregiver computer, a patient smartphone, and a remote patient database.

6. The smart pill organizer device of claim 1, wherein said first sensor comprises: an electromagnetic sensor; a RFID or NFC sensor; a capacitance sensor; or a load sensor.

7. The smart pill organizer device of claim 6, wherein the first sensor, or a wireless tagging sensor comprises a patient unique identifier on a carrier tray or on each of the plurality of dose units, able to detect when a dose is missed, an incorrect dose it taken, and trigger an audio, visual or wireless alarm emitted from a patient electronic computing device.

8. The smart pill organizer device of claim 7, wherein the smart pill organizer device also includes at least one wireless reader for said wireless tag sensors.

9. The smart pill organizer device of claim 6, wherein an electrical circuit is printed or overlaid onto each dose unit, at least a portion of the circuit being designed to be broken when the pills interior to the dose unit are removed from the dose unit, and wherein this disruption in the circuit or antenna is detectable, at least in part, by a capacitance or magnetic sensor connected to the microprocessor.

10. The smart pill organizer device of claim 9, wherein the electrical circuit is printed to have different combinations of resistance or capacitance between doses, the removal of a dose being measured by detecting the change in the circuit's resistance or capacitance.

11. The smart pill organizer device of claim 10, wherein the removable dose unit has a magnetic or capacitive material attached to the bottom of the dose unit.

12. The smart pill organizer device of claim 1, wherein the first sensor is a wireless tag reader that is connected to the printed circuit board, and wherein a wireless tag is also attached to each removable dose unit.

13. The smart pill organizer device of claim 12, wherein the wireless tag has a unique identification that correlates to a unique medication or combination of medications and a unique day, a dose time, and a dose window either in a local storage on the smart pill organizer device or in a cloud-based dose management system.

14. The smart pill organizer device of claim 1, further comprising at least one electrical and/or mechanical component for verifying the identity of a user before unlocking the pillbox container, comprising one or more of: a fingerprint scanner, a passcode reader, a two-factor authentication reader, and a voice recognition device.

15. The smart pill organizer device of claim 1, wherein said device is able to function as a hub in which the wireless radio is a cellular-enabled radio that is connected to one or more patient health monitoring devices via Bluetooth or WiFi, and able to wirelessly transmit a patient health reading to a caregiver computer, a patient smartphone, a remote patient database, and a doctor's office computer.

16. The smart pill organizer of claim 1, wherein the removable dose units are each about plus or minus 25% of: 1.4 inches wide, by 1.1 inches tall, by 1 inch deep.

17. A method of using a networked computer system comprising a smart pill organizer device to track a patient's adherence to taking medication on time, comprising:
1) providing the smart pill organizer device, comprising a pillbox container holding a plurality of individual removeable dose units fillable by a robotic system, the pillbox container comprising from top to bottom:
a) a removable carrier tray, with a plurality of holes that are sized to hold the plurality of individual dose units in a manner that a top of the dose unit is above the tray and a bottom of the dose unit is beneath the tray;
b) a bottom unit, comprising:
i. a bottom container comprising a plurality of top holes sized to fit the plurality of individual dose units when the carrier tray rests upon the bottom container;
ii. a button on the bottom container that is depressed by a user to manually reorder a prescription refill, and/or to reset the smart pill organizer device to restart dose adherence monitoring, and/or to initiate a call to a pharmacist or a medical provider;
iii. an exterior case sized to hold the bottom container;
c) the pillbox container further comprising:
i. at least one first sensor, one beneath each dose unit or one beneath all dose units, each sensor able to be activated and deactivated by removing one dose unit, or pills within the dose unit, from the container;
ii. a wireless radio capable of: transmitting a signal to a cellular network, and/or to a nearby WiFi router, and/or a remote computer, and/or a Bluetooth remote monitoring peripheral;
iii. a replaceable battery;
iv. a local computer memory;
v. at least one processor or a microprocessor;
vi. a printed circuit board connecting all electrical components within the pillbox container;
d) wherein the plurality of individual removable dose units, each unit filled with one or more medications, labelled, and sealed closed with: a paper or foil, or a cap that can be popped on/off, or a pivotable cap that is hinged at one edge to keep the lid attached;
e) wherein the smart pill organizer device is able to communicate with one or more electronic computing devices via a wireless network to track a patient's medication adherence, send alerts when doses are missed or a wrong dose taken, and order refills automatedly and manually;
f) wherein the plurality of individual removable dose units are derived from a sheet of dose units that are able to be filled by a robotic system, tagged with a patient specific unique identifier, and separated into the individual dose units.
2) affixing a tagging sensor to the dose units, or on a sheet of dose units, or the tray, wherein the tagging sensor encodes a unique identifier assigned to a patient;
3) detecting by said smart pill organizer device, and/or by an electronic computing device, that a patient has missed taking a dose of medication on time, or taken an improper dose; and,
4) sending an alarm to one or more of the electronic computing device to remind the patient to take the dose, or that an improper dose was taken.

18. (canceled)

19. The method of claim 17, wherein said smart pill organizer device is able to wirelessly communicate with at least one remote computer or electronic computing device comprising: a pharmacy computer, a caregiver computer, a patient smartphone, remote patient database.

20. The method of claim 17, wherein said first sensor comprises: an electromagnetic sensor; a RFID or NFC sensor; a capacitance sensor; or a load sensor.

21. The method of claim 17, wherein the smart pill organizer device further comprises one or more of: a second sensor, able to be activated by the opening and closing of the lid; an accelerometer, which detects motion; and a gyroscope, which detects orientation towards the gravity vector.

22. The method of claim 17, wherein the first sensor is a wireless tag reader that is connected to the printed circuit board, and a wireless tag is attached to each removable dose unit, and able to detect when a dose is missed, an incorrect dose it taken, and trigger an audio, visual or wireless alarm emitted from a patient electronic computing device.

23. The method of claim 17, wherein the smart pill organizer device further comprises a lid, and comprises a touchscreen and a camera that are embedded into the lid or into the removable carrier tray or the bottom unit, and enabled for conducting wireless communications with a remote healthcare provider or a caretaker.

24. A computer program product to monitor medication adherence, comprising:
a) a non-transitory computer-readable storage device having computer-readable program instructions embodied thereon that when executed by a computer cause the computer to provide a refill, the computer-executable program instructions comprising:
b) computer program instructions to receive, from a smart pill organizer device, adherence data for a user;
c) computer program instructions to determine a dose count from the received adherence data;
d) computer program instructions to compare the determined dose count to one or more management parameters, wherein the one or more management parameters are determined based on one or more received product characteristics and wherein the one or more received product characteristics identify the product;
e) computer program instructions to determine, based on the comparison of the determined dose count to the one or more management parameters;
f) wherein when a dose count indicates that a dose is not taken on time, then an alert electronic message is wirelessly transmitted to one or more electronic computing devices comprising: a patient's, a pharmacy's, a caregiver's, and a doctor's office;
g) wherein when a dose count is taken, an electronic message comprising the time the dose was taken is wirelessly transmitted to the one or more electronic computing devices;
h) wherein the smart pillbox container comprises: a plurality of individual removeable dose units, derived from a sheet of dose units that are able to be filled by a robotic system, tagged with a patient specific unique identifier, separated into the individual dose units, the pillbox container comprising from top to bottom:
i. a removable carrier tray, with a plurality of holes that are sized to hold the dose units in a manner that a top of the dose unit is above the tray and a bottom of the dose unit is beneath the tray;
ii. a bottom unit, comprising:
a bottom container comprising a plurality of top holes sized to fit the dose units when the carrier tray rests upon the bottom container;
a button on the bottom container that is able to be depressed by a user to manually reorder a prescription refill, and/or to reset the device to restart dose adherence monitoring, and/or to initiate a call to a pharmacist or a medical provider;
an exterior case sized to hold the bottom container;
j) the pillbox container further comprising:
i. at least one first sensor, one beneath each dose unit or one beneath all dose units, each sensor able to be activated and deactivated by removing one dose unit, or pills within the dose unit, from the container;
ii. a wireless radio(s), capable of: transmitting a signal to a cellular network, and/or to a nearby WiFi router, and/or a remote computer, and/or a Bluetooth remote monitoring peripheral;
iii. a replaceable battery;
iv. a local computer memory;
v. at least one microprocessor;
vi. a printed circuit board connecting all electrical components within the pillbox container;
vii. wherein the smart pill organizer device is able to communicate with one or more electronic computing devices, using the computer program product, and via a wireless network to track a patient's medication adherence, send alerts when doses are missed or the wrong dose taken, and order refills automatedly and manually;
viii. wherein the plurality of individual removable dose units, each unit filled with one or more medications, labelled, and sealed closed with: a paper or foil, or a cap that can be popped on/off, or a pivotable cap that is hinged at one edge to keep the lid attached; and,
ix. wherein the plurality of individual removable dose units are derived from a sheet of dose units that are able to be filled by a robotic system, tagged with a patient specific unique identifier, and separated into the individual dose units.

25. (canceled)

26. The computer program product of claim 24, wherein the smart pill organizer device further comprises a lid, and comprises a touchscreen and a camera that are embedded into the lid or into the removable carrier tray or the bottom unit, and enabled for conducting wireless communications with a remote healthcare provider or a caretaker.

27. The computer program product of claim 24, wherein said first sensor comprises: an electromagnetic sensor; a RFID or NFC sensor; a capacitance sensor; or a load sensor.

28. The computer program product of claim 24, wherein the first sensor is a wireless tag reader that is connected to the printed circuit board, and a wireless tag is attached to each removable dose unit, and able to detect when a dose is missed, an incorrect dose it taken, and trigger an audio, visual or wireless alarm emitted from a patient electronic computing device.

29. The computer program product of claim 24, wherein based on a first sensor data, the microprocessor determines whether a refill of the medication is needed based on comparing a remaining pill quantity to a future dose schedule; and, wirelessly orders a refill request.

30. The computer program product of claim 24, wherein the smart pill organizer device further comprises a button that is able to be depressed by a user when a new carrier tray has been placed into the exterior case of the smart pill organizer; and/or to order a medication refill.

31. A method of filling a smart pill organizer device, comprising the steps of:
a) providing a plastic sheet of dimensions of about: 7 inches by 4 inches, or 8 inches by 4 inches, and having a plurality of dose units performed in the plastic sheet or able to be formed by a robotic machine;
b) loading a computer database on the robotic machine with a patient data comprising one or more of: a patient name, a medication type, a dose amount, and a dose time;
c) placing the plastic sheet in the robotic machine, and automatedly filling the dose units with one or more medications per the patient data;
d) printing a patient label and applying said label to a top surface of the plastic sheet;
e) cutting the plastic sheet into a plurality of individual dose units;
f) loading the individual dose units into a carrier tray, and shipping to a patient; and
g) loading the carrier tray with the individual dose units into the smart pill organizer device of claim 1.

32. The method of filling of claim 31, wherein the dose units are each about plus or minus 25% of: 1.4 inches wide, by 1.1 inches tall, by 1 inch deep.

33. (canceled)

34. (canceled)

35. The method of filling of claim 31, wherein the smart pill organizer device further comprises a lid, and comprises a touchscreen and a camera that are embedded into the lid or into the removable carrier tray or the bottom unit, and enabled for conducting wireless communications with a remote healthcare provider or a caretaker.

36. The method of filling of claim 31, wherein said first sensor comprises: an electromagnetic sensor; a RFID or NFC sensor; a capacitance sensor; or a load sensor.

37. The method of filling of claim 31, wherein the patient label is placed on each of the plurality of individual removable dose units and identifies the type and amount of each medication stored therein, and a patient name.
